(19) United States
(12) Patent Application Publication
(10) Pub. No.: US 2014/0018432 A1
(43) Pub. Date: Jan. 16, 2014

(54) ELECTROCHEMICAL GENERATION OF CHLORINATED UREA DERIVATIVES

(71) Applicant: Hercules Incorporated, Wilmington, DE (US)

(72) Inventor: Davit E. Sharoyan, Hockessin, DE (US)

(21) Appl. No.: 13/910,231

(22) Filed: Jun. 5, 2013

Related U.S. Application Data
(60) Provisional application No. 61/670,642, filed on Jul. 12, 2012.

(51) Int. Cl. C07C 273/18

(52) U.S. Cl. CPC C07C 273/1863
USPC 514/588; 564/32

(57) ABSTRACT

Method of single step electrochemical generation of chlorinated urea, chlorinated dimethylurea and other chlorourea derivatives is disclosed. The chlorinated species are generated in situ and upon demand and can be used for microbial control in industrial water treatment.

This application claims the benefit of U.S. Patent Application No. 61/670,642, Filed 12 Jul. 2012, the entire contents of which are hereby incorporated by reference.

### FIELD OF THE INVENTION

The present invention provides a convenient and easy method for electrolytic generation of halogenated products of urea and its derivatives, N-chlorourea and N-chloro-N,N′-dimethylurea in particular.

### BACKGROUND OF THE INVENTION

Haloamines are well known biocides which effectively reduce, inhibit and/or control the proliferation of microorganisms that cause biological fouling in circulating water. Haloamines biocides are typically generated by combining a solution of active halogen donor species (e.g., hypochlorite) with an amine-containing composition (e.g., an ammonium halide solution). For example, U.S. Pat. No. 5,976,386 and U.S. Pat. No. 6,132,628 by Barak disclose the preparation of haloamine biocides from hypochlorite and various ammonium salts for use in treating liquids to inhibit the growth of microorganisms. As described in the other patents, e.g. U.S. Pat. No. 3,328,294 by Self or U.S. Pat. No. 5,565,109 by Sweeny, haloamines can be formed by combining hypochlorite with a source of organic or inorganic amine. Stability and biocidal activity of these species varies. US2010/0331416 by Jerusik describes the method of generation of N-chloro-urea, N-chloro-N,N′-dimethylurea and other modified chloro ureas by the addition of sodium hypochlorite (bleach) to a solution containing urea or dimethylurea.

In these patents, the hypochlorite solution is not generated in situ, but is instead taken from a reservoir of pre-existing solution. These active halogen donor species, such as hypohalites, are strong, corrosive oxidants, making them both difficult and dangerous to handle, especially in large quantities. Furthermore, these species degrade over time, resulting in active halogen donor species solutions having decreased potency and efficacy.

A number of patents and publications from prior art describe the generation of haloamines by electrochemical method. For example, C. Trembley et al., J. Chim. Phys., 90, 79 (1993). C. Trembley et al., J. Chim. Phys., 91, 535 (1994). or B. V. Lyalin, et al., Russian Chem. Bull., 47, 1956 (1998) described electrochemical generation of monochloroamine (NH₂Cl) in one step from ammonia in aqueous halide salt solution. These attempts resulted in low yields of monochloramine. Lyalin also discloses a two-step preparation of NH₂Cl in 50% overall yield. A solution of NCl₃ in carbon tetrachloride is electrochemically generated from NH₄Cl in one apparatus. This NCl₃solution is then mixed with ammonia in a second apparatus to generate NH₂Cl. US2008/18185 by Cheng also describes a two step process wherein the active chlorine species are generated by electrochemical method at first and then are combined with ammonium or amine source to make chloramine species on demand.

WO2006/103314 by Savolainen describes the method for electrochemical generation of microbiocidal solutions by passing solutions through a cell divided by a membrane. The original solutions contain sodium, ammonium, chloride, bromide and other ions. The resulting anolite and catholite solutions can be used separately or in combined form for disinfection, sterilization, prevention of bacterial growth and/or prevention of biofilms.

U.S. Pat. No. 3,776,825 to Jaroslav discloses aqueous monohaloamine solutions generated in an electrochemical cell charged with a halide salt solution and an amine containing compound for use in dental applications. Active halogen donor species are electrochemically generated and converted to hypohalite in the presence of hydroxide ions. The hypohalite reacts in situ with the amine containing compound to form monohaloamine. Similarly, in Russian Journal of Electrochemistry, vol. 36, No 11, 2000, Lyalin et al describe the generation of chlorinated derivatives of arylsulfonamides by electrolysis of solutions containing sodium chloride and arylsulfonamide source.

### SUMMARY OF THE INVENTION

The present invention relates to the electrochemical generation of chlorinated urea and chlorinated dimethylurea derivatives in a single step reaction by subjecting solutions containing a chloride source and urea or dimethylurea to electrolysis. The single step reaction is the combination of the electrolysis and the chlorination of the urea. Upon electrolysis active chlorine species (e.g. sodium hypochlorite or hypochlorous acid) are formed in situ that immediately react with the amine groups of urea or dimethylurea forming chlorinated derivatives. The method provides for increased yields and stability of the chlorinated products.

A method of generation of chlorinated urea or chlorinated urea derivatives is disclosed. The method comprises:
i) charging an electrochemical cell with a chloride solution containing a) a chloride source; b) urea, urea derivative, or combinations thereof; and c) acid;
ii) subjecting the solution to electrolysis in the electrochemical cell and generating at least one active halogen donor species;
iii) allowing the at least one active halogen donor species to react with the urea, urea derivative, or combinations thereof, in the solution to produce a chlorinated urea or a chlorinated urea derivative in situ.

The chloride source is a soluble inorganic chloride. Examples include, but are not limited, to sodium chloride, potassium chloride, lithium chloride, hydrochloric acid and combinations thereof.

In one embodiment the urea derivative comprises N,N′-dimethylurea.

In one embodiment the chlorinated urea derivative comprises N-chloro-N,N′-dimethylurea.

The acid can comprise phosphoric acid.

The pH of the solution in iii) can be less than or equal to 8, and can be less than 7.

The pH of the initial solution containing dimethylurea, soluble chloride, and add prior to the electrolysis can be in the range of from about 1 to 8, and can be a pH of from about 1 to about 7, or from about 1 to about 5, and may be a pH of about 1 to about 3.

The pH of the final solution after the electrolysis containing N-chloro-N,N′-dimethylurea derivative can be in the range of from about 5 to 8.

The electrochemical cell can be a flow cell or batch cell,

A method of treating a liquid to control microbial growth is also disclosed. The method comprises the step of addition of the chlorinated urea, or chlorinated N,N′-dimethylurea, or other chlorinated urea derivatives, or a mixture thereof prepared according to the method above, to the liquid in an amount effective to reduce, control and/or inhibit the growth of microorganisms within,

### DETAILED DESCRIPTION OF THE INVENTION

The method consists of preparation of aqueous solution containing a chloride source and urea or urea derivative (e.g. N,N′-dimethylurea) and subjecting it to electric current in a flow cell. Upon electrolysis active chlorine species (molecular chlorine, hypochlorous acid or hypochlorite) are generated in situ. These species immediately react with urea or urea derivative and generate chlorinated species. For higher yields and better stability, the chloride and urea or urea derivative solution is acidified before electrolysis. Acid is added for neutralization of the base resulting from electrolysis and for stabilization of the final product solution.

In addition, the present invention is directed to the methods of controlling microbial populations in industrial process waters by administering effective amounts of the N-chlorourea and N-chloro-N,N′-dimethylurea. Electrolytic chlorination of urea and N,N′-dimethylurea as described below provides an alternative method for production of a biocide.

According to the present invention, chloro-urea or chlorinated urea derivatives (chlorinated dimethylurea in particular) can be prepared by using an electrochemical cell wherein the active chlorine species are electrochemically generated in situ upon demand. Thus, the degradation, handling, transportation and safety problems are minimized since reservoirs of active halogen donor species solutions do not have to be filled and maintained over a period of time.

The present invention discloses an electrochemical method for generation of N-chlorourea (CU) and chlorinated products of urea derivatives, specifically N-chloro-N,N′-dimethylurea (DMCU). A method of generation of chlorinated urea or chlorinated urea derivatives is disclosed. The method comprises:
i) charging an electrochemical cell with a solution containing a) a chloride source; b) urea, urea derivative, or combinations thereof; and c) acid;
ii) electrochemically generating at least one active halogen donor species;
iii) wherein the at least one active halogen donor species reacts with the urea, urea derivative, or combinations thereof, in the solution to produce a chlorinated urea or a chlorinated urea derivative in situ.

Also disclosed is a method of generation of chlorinated N,N′-dimethylurea comprising:
i) charging an electrochemical cell with a chloride solution containing a chloride source and N,N′-dimethylurea;
ii) electrochemically generating at least one active halogen donor species;
iii) wherein the at least one active halogen donor species reacts N,N′-dimethylurea in the solution to produce a chlorinated N,N′-dimethylurea in situ.

The chloride source can be a soluble inorganic chloride. Examples include, but are not limited to, sodium chloride, potassium chloride, lithium chloride, calcium chloride, magnesium chloride, hydrochloric acid and combinations thereof.

In one embodiment the urea derivative comprises N,N′-dimethylurea.

In one embodiment the chlorinated urea derivative comprises N-chloro-N,N′-dimethylurea.

In one embodiment the chlorinated urea is N-chlorourea.

The acid source is any acid that will adjust the pH of the solution. The acid can be an acid that provides buffering. One example of a suitable acid is phosphoric acid. Other example acids are hydrochloric or sulfuric.

In some embodiments the molar ratio of chloride to urea can be 10:1 to 1:1, can be from 10:1 to 3:1 and may be from 10:1 to 5:1. Although a ratio of greater than 10:1 of chlorine to urea can be used there is no added benefit.

Current efficiency of urea and dimethylurea electrochlorinations varied from 7% to 75% depending on flow rate, loads of urea or urea derivative, temperature and other factors. It was significantly higher for electrogeneration of N-chloro-N,N′-dimethylurea as compared to that of N-chlorourea. Voltage between electrodes needs to be sufficient for 2Cl⁻-->Cl₂oxidation process, for example 1.5 volts or greater and can be 2.0 volts or higher. Current density and the surface area of electrodes define the amount of active chlorine species generated within a unit of time.

Concentration of the chloride source can range from between 0.3% to 5.0%, can be from 0.5 to 3.0%, can be 0.5% to 1.0%, and may be 0.5% to 0.9% by weight percent.

Although the process can be run at high concentrations of chloride ion, it can be accomplished at lower levels so to minimize corrosion in the electro-generation system. The chloride molar concentration can be less than 1.0 molar, can be less than 0.75, can be less than 0.5, can be less than 0.15, but greater than 0.05 molar.

The pH of the initial solution of step i), prior to reaction in the electrochemical cell can be in the range of from about 1 to about 7, and may be from about 1 to about 3.

The solution reaction product in iii) is most stable in acidic to near neutral conditions, such as, a pH of 8 or less and can be about 7 or less. The pH of the solution in iii) can be less than or equal to 8 and may be less than about 7. If the pH of the solution is not in the range of about 5 to 8 after iii) it can be adjusted to a pH of about 5 to 8 by addition of an acid prior to addition to a water system. This pH range helps to minimize or prevent corrosion of the system being treated.

The pH of the final solution after the electrolysis of a solution containing N-chloro-N,N′-dimethylurea derivative can be in the range of from about 5 to 8. Lower pH values are acceptable. However, prior to adding the biocide to the water to be treated the pH can be adjusted to between about 5 and 8.

The electrochemical cell can be a flow cell or batch cell.

Also disclosed is a method of treating a liquid to be treated for microbial growth comprising the step of adding the chlorinated urea, or chlorinated N,N′-dimethylurea, or other chlorinated urea derivatives, or a mixture thereof to the liquid to be treated in an amount effective to reduce, control and/or inhibit the growth of microorganisms within.

The concentration of the chlorinated urea, or chlorinated N,N′-dimethylurea, or other chlorinated urea derivatives or mixtures thereof used to treat liquid is at least 1.0 ppm. However, the concentration can be from 0.1 to 200 ppm, can be from 0.1 to 50 ppm, and may be from 0.01 to 10 ppm.

### Electrogeneration of N-Chlorourea

For generation of chlorourea by this method an aqueous solution containing a chloride source, for example sodium chloride or hydrochloric acid, and urea are subjected to an electric current. Electrolysis of a solution, consisting of a chloride source and urea leads to a formation of N-chlorourea. For example using sodium chloride and urea the reaction would be:

2NaCl+H₂O→NaOCl+NaCl+H₂

NaOCl+H₂NCONH₂→H₂NCONHCl+NaOH

It has been found that inclusion of a membrane between electrodes during batch electrolysis increased the yields significantly. Particularly, preferred membranes are those that allow the flow of cations through the membrane but not anions and electrons. An example of such a membrane is the Nafion™ (E. I. du Pont de Nemours and Company, Wilmington, Del.) which are made from special fluorinated copolymers which contain sulfonate groups. In some examples the yield increase was more than 50%, could be more than 75%, and may be more than a 100% increase as compared with the electrolysis conducted without the membrane. It is theorized that membranes protect chlorinated urea species from the reduction on the cathode surface. Acidification of urea solutions also improves yield as it slows down the decomposition of chlorinated products.

The present invention provides for improved yields on chlorourea generation.

Chlorinated urea species are known for their instability. For that reason chemical and electrochemical oxidation of urea has been reported as one of the methods to remove urea from aqueous media (e.g. as reported in AIChE Journal vol. 32, No 9, 1986). According to the reference, the electrolytic oxidation of solutions containing sodium chloride and urea results in formation of N₂, O₂, CO₂. and H₂ gases as products.

N-chloro-N,N′-dimethylurea appears to be significantly more stable than N-chlorourea and it can be produced at significantly higher yields and current efficiency using the present invention.

### Generation of N-Chloro-N,N′-Dimethylurea

For generation of DMCU by this method an aqueous solution containing a chloride source, for example sodium chloride or hydrochloric acid, dimethylurea (DMU) and acid, such as phosphoric acid, are subjected to an electric current. Electrolysis of a solution, consisting of dimethylurea, and a chloride source, such as sodium chloride, proceeds easily and leads to formation of N-chloro-N,N′-dimethylurea:

2NaCl+H₂O →NaOCl+NaCl+H₂

NaOCl+MeHN—C(O)—NHMe→MeHN—C(O)—NClMe+NaOH

If electrolysis is carried out in a batch mode in a cell with a membrane, N-Chloro-N,N-dimethylurea can be generated in high yield. In some examples the yield increase was more than 50%, could be more than 75%, and may be more than 100% when compared with the electrolysis conducted without the membrane. The membranes allow the flow of cations but not anions. An example of one such membrane is a Nafion membrane. In the flow cell without separation between electrodes yields are still significant.

In addition to the chlorinated product DMCU, electrolytic process generates equimolar amount of base NaOH. As a result pH of the solution can increase from the neutral 7.3 to highly basic 12.5 if not controlled. The presence of a membrane between the electrodes keeps the pH of anolite solution acidic. The process is not affected by the formation of NaOH around cathode.

In the flow cell, however there is no any separation membrane between electrodes so the pH of the solution can easily increase to 12 and higher if not controlled. R was found that basic solutions of N-chloro-N,N′-dimethylurea are not stable and significant amounts of DMCU decompose within short period of time.

Addition of acids, e.g. phosphoric acid can significantly improve yields of N-Chloro-N,N-dimethylurea in the electrochlorination process. That is because in the presence of phosphoric add pH change is controlled by the formation of sodium dihydrogen phosphate/disodium hydrogen phosphate buffer:

NaOCl+MeHN—C(O)—NHMe+H₃PO₄→MeHN—C(O)—NClMe+NaH₂PO₄

2NaOCl+2MeHN—C(O)—NHMe+H₃PO₄→2MeHN—C(O)—NClMe+Na₂HPO₄

To avoid DMCU decomposition it is desirable to keep pH levels below 8. In neutral or acidic environment DMCU solutions are significantly more stable. The pH can be controlled using addition of phosphoric add or other add.

Electrochemical chlorination of urea and its analogues can be implemented by using hypochlorite generators available for on-site generation of dilute bleach (up to 8,000 ppm of active chlorine species).

Electrochemical generation of DMCU has a number of advantages compared to conventional synthetic method from bleach and dimethylurea. By utilizing on-site electrochlorination method issues related to transportation, storage and handling corrosive chemicals like bleach are completely eliminated. The chemicals used in the process are safe and easy to handle. DMCU can be produced right before its use and in the desirable amounts.

Microbiological studies indicate that both N-chlorourea, N-chloro-N,N′-dimethylurea and other chlorinated derivatives of modified ureas are potent biocides. Therefore persons skilled in the art will recognize that the processes of the present invention can be used for microbial control in a wide range of applications including and not limited to industrial water treatment, cooling water towers, paper and pulp production, disinfection of swimming pools, municipal water treatment, food treatment, and pharmaceutical applications.

The present invention will now be described with reference to a number of specific examples that are to be regarded as illustrative and not restricting the scope of the present invention.

### EXAMPLES

In the electrochemical method of generation of N-Chlorourea and N-Chloro-N,N′-dimethylurea (DMCU), an aqueous solution containing sodium chloride, as the chloride source, and urea, or sodium chloride (chloride source) and diniethylurea (and phosphoric acid) have been subjected to an electric current.

Experiments have been run in both flow and batch mode. Flow cells, ESR 160 and BMSC-13 from Davey Water Products have been used for electrochlorination in flow mode. These units are able to produce about 1 lb/day as 100% dry chlorine equivalent. The maximum current in the system is 15.5 Amp for ESR 160 and about 12.5 Amp for BMSC-13. The salt concentration for electrochlorination is specified to be between 3000 and 7000 ppm. Electroplates are stacked parallel to each other with anodes covered with ruthenium dioxide coating. Experiments have been run at room temperature. Haloamine concentrations have been determined using UV-VIS and NMR spectroscopy and in some cases by a Hach test kit.

Electrochemical chlorinations in batch mode have been carried out using equipment from BAS Analytical, including BAS Epsilon, PWR-3 power booster and electrolytic cell with 100 mL volume (see Examples 1 and 2). Titanium electrodes with special ruthenium dioxide coating (RuO₂/Ti) have been used as the anode for electrochemical generation of active chlorine donor species. A platinum coil has been used as a cathode. A specially designed barrier has been made from Nafion™ membrane and placed between electrodes in divided electrochemical cells.

Electrochemical generations of active chlorine donor species in batch have been conducted at 2.0 V potential (relative to Ag/AgCl reference electrode where E_{Ag/AgCl}=0.196 V).

Electrochemical generation of all active halogen donor species has been carried out in an ice/water bath at 0° C. Unless otherwise stated, aliquots of anode chamber solution have been removed every 10 or 20 minutes for 2 hours to determine concentration of active halogen donor species and pH. Haloamine concentrations have been determined using UV-VIS spectroscopy and in some cases by a Hach test kit.

### Example #1

The undivided cell was charged with 100 ml solution containing 30,000 ppm (0.513 M) sodium chloride and 10,000 ppm (0.167 M) urea. The solution was acidified to pH 2.8 and then subjected to electrolysis by passing 1 Amp current through the solution for one hour. The electrolysis has produced solution containing 3460 ppm (0.036 M) of chlorinated urea (CU) (in 21% yield and 18% current efficiency).

When the solution containing 25,000 ppm sodium chloride (0.427 M) and 10,000 ppm (0.167 M) urea was electrolyzed in the divided cell with Nafion™ membrane separator between electrodes, the electrolysis has produced a solution containing 9,050 ppm (0,096 M) of CU (in 57% yield and 47% current efficiency) within 60 minutes. The identities of the products were confirmed by observing a band at 252 nm in UV-VIS spectra and NMR analyses.

### Example #2

The undivided cell was charged with 100 ml solution containing 25,000 ppm (0.427 M) sodium chloride and 10,000 ppm (0,114 M) N,N′-dimethylurea. The solution was subjected to electrolysis by passing 1 Amp current through the solution for one hour. The electrolysis has produced solution containing 5,300 ppm (0.043 M) DMCU in 38% yield and 27% current efficiency within 60 minutes.

When the same solution was electrolyzed in the divided cell with Nafion™ membrane separator between electrodes, the electrolysis has produced a solution containing 12,400 ppm (0.101 M) DMCU (in 89% yield and 99% current efficiency) within 30 minutes. The identity of the product and its concentrations were monitored by UV-VIS and NMR analyses.

### Example #3

Aqueous solution containing 7000 ppm (0.120 M) sodium chloride, 2500 ppm urea (0.042 M) and 1250 ppm phosphoric acid (0.013 M) was subjected to electrolysis in ESR 160 cell in a single pass mode with a flow rate of 0.1 L/min. The electrolysis has generated a steady flow of 230 ppm (0.002 M) of chlorourea, CU (in 6% yield and 7% current efficiency). The identity of the chlorinated product was confirmed by NMR and UV-VIS spectra. Hydrogen gas which has been vented out from the system. The pH of the final solution has changed from 2.1 to 2.6.

### Example #4

Aqueous solution containing 7000 ppm (0.120 M) sodium chloride, 2500 ppm DMU (0.028 M) and 1250 ppm phosphoric acid (0.013 M) was subjected to electrolysis in ESR 160 cell in a single pass mode with a flow rate of 0.1 L/min. The electrolysis has generated a steady flow of 2370 ppm (0.019 M) of DMCU (in 68% yield and 45% current efficiency). The identity of the product and its concentrations were confirmed by UV-VIS (band at 262 nm) and NMR analyses. The process has also produced hydrogen gas which has been vented out from the system. The pH of the final solution has increased from 2.1 to 6.9 and stabilized at that point.

### Example #5

Aqueous solution containing 7000 ppm (0.120 M) sodium chloride, 5000 ppm DMU (0.057 M) and 1250 ppm phosphoric acid was subjected to electrolysis in ESR 160 cell in a single pass mode with a flow rate of 0.1 L/min. The electrolysis has generated a steady flow of 1800 ppm (0.015 M) of DMCU (in 26% yield and 31% current efficiency). The concentrations of the product were determined by UV-VIS and NMR analyses. Hydrogen gas was vented out from the system. In this test pH of the solution has increased from 2.1 to 6.1.

### Example #6

Aqueous solution containing 7000 ppm (0.120 M) sodium chloride, 1250 ppm DMU (0.014 M) and 625 ppm phosphoric acid was subjected to electrolysis in BMSC-13 cell in a single pass mode with a flow rate of 0.2 L/min. The electrolysis has generated a steady flow of 1300 ppm (0.011 M) of DMCU (in 75% yield and 55% current efficiency). Hydrogen gas was vented out from the system. In this test pH of the solution has increased from 2.2 to 7.1.

### Example #7

Aqueous solution containing 7000 ppm (0.120 M) sodium chloride, 3750 ppm DMU (0.043 M) and 1875 ppm phosphoric acid was subjected to electrolysis in BMSC-13 cell in a single pass mode with a flow rate of 0.05 L/min. The electrolysis has generated a steady flow of 2600 ppm (0.021 M) of DMCU (in 49.8% yield and 28% current efficiency). Hydrogen gas was vented out from the system. In this test pH of the solution has increased from 1.9 to 6.2.

### Example #8

Aqueous solution containing 7000 ppm (0.120 M) sodium chloride, 1250 ppm DMU (0.014 M) and 500 ppm phosphoric acid was subjected to electrolysis in BMSC-13 cell in a single pass mode with a flow rate of 0.30 L/min. The electrolysis has generated a steady flow of 1130 ppm (0.09 M) of DMCU (in 65.0% yield and 69% current efficiency). Hydrogen gas was vented out from the system. In this test pH of the solution has increased from 2.3 to 6.9.

### Example #9

Aqueous solution containing 7000 ppm (0.120 M) sodium chloride, 1250 ppm DMU (0.014 M) and 500 ppm phosphoric acid was prepared by dissolving solid sodium chloride, dimethylurea and 85% phosphoric acid in deionized water in the mixing tank. Then solution was pumped through and subjected to electrolysis in ESC Max 50 cell in a single pass mode. A flow rate for the electrolyzed solution was varied from 0.8 to 1.4 L/min. The product solution was collected in 4L separation flask where it was degassed and then passed into a product storage tank. The amounts of DMCU generated by electrolysis were measured by UV-VIS and ¹H NMR spectroscopy, In these tests pH of the solution had increased from the original 2.3 to the final 6.5-7.8 depending on flow rate. The percent chlorination of DMU was also dependent on solution flow rate and increased from 47% to 77% upon slowing the solution flow rate from 1.4 L/min to 0.8 L/min.

1. A method of generation of chlorinated urea or chlorinated urea derivatives comprising:
i) charging an electrochemical cell with a chloride solution containing a) a chloride source; b) urea, urea derivative, or combinations thereof; and c) acid;
ii) electrochemically generating at least one active halogen donor species;
iii) wherein the at least one active halogen donor species reacts with urea, urea derivative, or combinations thereof, in the solution to produce a chlorinated urea or a chlorinated urea derivative in situ.

2. The method of claim 1, wherein the chloride source is a soluble inorganic chloride

3. The method of claim 2, wherein the chloride source is selected from the group consisting of sodium chloride, potassium chloride, lithium chloride, hydrochloric acid and combinations thereof.

4. The method of claim 1, wherein the urea derivative comprises N,N′-dimethylurea.

5. The method of claim 3, where in the acid comprises phosphoric acid.

6. The method of claim 1, wherein the chlorinated urea derivative comprises N-chloro-N,N′-dimethylurea.

7. The method of claim 1, wherein the pH of the solution in iii) is less than or equal to 7.

8. The method of claim 1, wherein the acid comprises phosphoric acid.

9. The method of claim 1, wherein the pH of the initial chloride solution containing dimethylurea, soluble chloride, and acid prior to the electrolysis of step ii) is from about 1 to 7.

10. The method of claim 9, wherein the pH of the initial chloride solution is from about 1 to 3.

11. The method of claim 4, wherein the pH of the final solution after the electrolysis containing N-chloro-N,N′-dimethylurea derivative is from about 5 to 8.

12. The method of claim 1, wherein the electrochemical cell is a flow cell.

13. The method of claim 1, wherein the electrochemical cell is a batch cell.

14. The method according to claim 1, wherein the electrochemical cell has a voltage of 1.5 or higher.

15. The method according to claim 1, wherein the chloride source is from about 0.3% to about 6.0% of the chloride solution.

16. A method of treating a liquid comprising the step of addition of the chlorinated urea, or chlorinated N,N′-dimethylurea, or other chlorinated urea derivatives, or a mixture thereof prepared according to the method of claim 1 to the said liquid in the amount effective to reduce, control and/or inhibit the growth of microorganisms within.

17. A method of generation of chlorinated N,N′-dimethylurea comprising:
i) charging an electrochemical cell with a chloride solution containing a chloride source and N,N′-dimethylurea;
ii) electrochemically generating at least one active halogen donor species;
iii) wherein the at least one active halogen donor species reacts with N,N′-dimethylurea in the solution to produce a chlorinated N,N′-dimethylurea in situ.

18. The method of claim 15, wherein the chloride source is a soluble inorganic chloride selected from the group consisting of sodium chloride, potassium chloride, lithium chloride, hydrochloric acid and combinations thereof.

19. A method of treating a liquid comprising a step of addition of the chlorinated N,N′-dimethylurea prepared according to the method of claim 15, to the liquid in an amount effective to reduce, control and/or inhibit the growth of microorganisms within.
